(19) United States
(12) Patent Application Publication
(10) Pub. No.: US 2012/0065158 A1
(43) Pub. Date: Mar. 15, 2012

(54) FUCOIDAN HAVING ANTITUMOR ACTIVITY

(76) Inventors: Yoshiharu Okamoto, Tottori (JP); Saburo Minami, Tottori (JP); Takeshi Tsuka, Tottori (JP); Yasunari Miki, Tottori (JP)

(21) Appl. No.: 13/258,108

(22) PCT Filed: Mar. 19, 2010
(86) PCT No.: PCT/JP2010/054870

§ 371 Date: Nov. 28, 2011

(30) Foreign Application Priority Data
Mar. 23, 2009 (JP) 2009-069804

(51) Int. Cl. A61K 31/737; A61P 35/00; C07H 5/10

(52) U.S. Cl. 514/54; 536/122

(57) ABSTRACT

Disclosed is a medium-molecular weight fucoidan which can be used in treating and/or preventing proliferative diseases, in particular, cancer. Specifically disclosed are a medium-molecular weight fucoidan, a method for producing the same, a medicinal composition containing the same, and use of the medium-molecular weight fucoidan in producing a drug for treating and/or preventing preventing proliferative diseases, in particular, cancer. The aforesaid medium-molecular weight fucoidan has an average molecular weight of about 6,000 to about 2,000,000, preferably an average molecular weight of about 40,000 to about 330,000, more preferably an average molecular weight of about 40,000 to about 244,000, and still more preferably an average molecular weight of about 80,000 to about 200,000.

### TECHNICAL FIELD

The present invention relates to a fucoidan useful for treating and/or preventing a proliferative disease, in particular, a cancer. More specifically, the invention relates to a medium-molecular-weight fucoidan, a process for producing the same, a pharmaceutical composition containing the same, and the use of a medium-molecular-weight fucoidan in the preparation of a medicament for treating and/or preventing a proliferative disease, in particular, a cancer.

### BACKGROUND ART

Fucoidan, which is a natural product, is a polysaccharide containing sulfated fucose, and can be extracted from for example marine algae or brown algae that grow naturally, such as okinawamozuku (Cladosiphon okamuranus), mozuku (Nemacystus decipiens), wakame (Undaria pinnatifida), mekabu (sporophyl of wakame), and kelp (Laminaria). It has been made evident that this polysaccharide has biological activities over a wide range, such as the extinction of cancer cells, the control of the immune system, and the promotion of the regeneration of organs. Moreover, fucoidan has an effect of contracting the skin, and has a moisturizing effect. Thus, fucoidan is also used as cosmetics. In this way, demands of fucoidan have been increasing for a raw material or component of healthy foods, functional foods, supplements, cosmetics, pharmaceutical products and the like.

Researches, in particular, about the antitumor effect of fucoidan have been made over many fields. For example, Patent Literature 1 (JP-A-2007-051081) discloses that acetylfucoidan or sulfated acetylfucoidan is useful, in particular, as an antitumor agent. However, it has been known that there are numbers of fucoidans which have various molecular weights respectively (see, for example, Patent Literature 2), for example, fucoidans ranging from molecular weights of several hundreds to several millions. In general, a low-molecular-weight fucoidan is used since this species is easily absorbable into a living body. However, it has not yet been studied systematically what size of fucoidan species has the antitumor effect.
Patent Literature 1: JP-A-2007-051081
Patent Literature 2: International Publication WO 2006/093175

### DISCLOSURE OF THE INVENTION

### Problems to be Solved by the Invention

An object of the invention is to provide a fucoidan, in particular, a medium-molecular-weight fucoidan having the excellent antitumor effect.

### Means for Solving the Problems

The inventors have repeatedly made intensive investigations to solve the problems. The inventors found that the antitumor effect of fucoidans having specific molecular weights is in particular excellent through systematic investigations about the antitumor effect using fucoidans with different molecular weights. Furthermore, the inventors have found that a medium-molecular-weight fucoidan has an excellent antitumor effect. Additionally, the inventors found that the medium-molecular-weight fucoidan can be produced effectively by treating a raw fucoidan as a natural product under hydrothermal conditions. On the basis of these findings, the inventors accomplished the invention.

Accordingly, the medium-molecular-weight fucoidan according to the invention denotes a fucoidan having an average molecular weight of about 6,000 to about 2,000,000, preferably about 40,000 to about 330,000, more preferably about 40,000 to about 244,000, even more preferably about 80,000 to about 200,000, in particular preferably about 130,000 to about 138,000.

In a preferred embodiment, the fucoidan of the invention is produced by extracting a crude fucoidan from a raw material, subjecting this fucoidan to hydrothermal treatment to be turned into a middle-molecular-weight molecule form, and purifying the resultant to provide a fucoidan having a desired molecular weight. Accordingly, the invention provides a medium-molecular-weight fucoidan that is useful for treating and/or preventing a proliferative disease, and is produced by hydrothermal treatment, so as to have one of the above-mentioned average molecular weight ranges.

In another aspect, the invention provides a process of producing a medium-molecular-weight fucoidan by hydrothermal treatment.

In a still another aspect, the invention provides a pharmaceutical composition, which is useful for treating and/or preventing a proliferative disease, comprising the medium-molecular-weight fucoidan of the invention as an effective component.

In a further aspect, the invention provides a food or drink, which is useful for treating and/or preventing a proliferative disease, comprising the medium-molecular-weight fucoidan of the invention.

In an additional aspect, the invention provides a method for treating and/or preventing a proliferative disease, comprising administering the medium-molecular-weight fucoidan of the invention in an effective amount to a subject for which the treatment and/or the prevention is required. The subject may be a mammal such as a human.

In additional aspects, the invention provides use of the medium-molecular-weight fucoidan of the invention in the preparation of a medicament for treating and/or preventing a proliferative disease; a method for treating and/or preventing a proliferative disease, comprising administering the medium-molecular-weight fucoidan of the invention in an effective amount to a subject for which the treatment and/or the prevention is required; and the medium-molecular-weight fucoidan of the invention for treating and/or preventing a proliferative disease.

### Advantages of the Invention

The medium-molecular-weight fucoidan supplied by the invention has a remarkable antitumor effect as compared with low-molecular-weight fucoidans, for example, a fucoidan having an average molecular weight less than about 6,000, and high-molecular-weight fucoidans, for example, a fucoidan having an average molecular weight of about 2,000,000 or more. Thus, the medium-molecular-weight fucoidan is useful as a pharmaceutical product. Furthermore, the medium-molecular-weight fucoidan of the invention is suitable for being used in a healthy food, functional food, supplement, cosmetic product or some other that is expected to produce a tumor-preventing effect.

### BRIEF DESCRIPTION OF THE DRAWINGS

FIG. 1 shows the respective average tumor weights of Colon-26-transplanted-mice after fucoidans having different molecular weights were each administered for 14 days. Each error bar shows the standard deviation (concerned). A significant verification was carried out by Tukey-Kramer's test (P<0.05).

FIG. 2 shows the respective average tumor growth rates of Colon-26-transplanted-mice after fucoidans having different molecular weights were each administered for 14 days. Each error bar shows the standard deviation. A significant verification was carried out by Tukey-Kramer's test.

FIG. 3 shows the respective survival periods (days) of Colon-26-transplanted-mice to which fucoidans having different molecular weights were each administered.

FIG. 4 shows the respective survival periods (days) of Colon-26-transplanted-mice to which fucoidans having different molecular weights were each administered.

FIG. 5 shows the respective survival periods (days) of Colon-26-transplanted-mice to which fucoidans having different molecular weights were each administered. Each error bar shows the standard deviation. A significant verification was carried out by Bonferroni/Dunn test (P<0.05).

### BEST MODE FOR CARRYING OUT THE INVENTION

Accordingly, a first aspect of the invention provides a medium-molecular-weight fucoidan useful for treating and/or preventing a proliferative disease. The medium-molecular-weight fucoidan according to the invention includes a fucoidan having an average molecular weight of about 6,000 to 2,000,000, preferably an average molecular weight of about 40,000 to about 330,000, more preferably an average molecular weight of about 40,000 to about 244,000, even more preferably an average molecular weight of about 80,000 to about 200,000, in particular preferably an average molecular weight of about 110,000 to about 138,000. The medium-molecular-weight fucoidan has an average molecular weight in this specified range, and the medium-molecular-weight fucoidan particularly has preferably an average molecular weight of about 40,000 to about 330,000, more preferably an average molecular weight of about 40,000 to about 244,000, even more preferably an average molecular weight of about 80,000 to about 200,000, in particular preferably an average molecular weight of about 130,000 to about 138,000. The medium-molecular-weight fucoidan is produced typically by treating a crude fucoidan to be converted into a medium-molecular-weight molecule form. In the invention, the treatment into the medium-molecular-weight molecule form may be conducted at the same time when fucoidan-extracting treatment from an original material or raw material is conducted; or may be conducted continuously after the extracting treatment or may be in an independent batch step after the extraction.

In a more specific embodiment, the fucoidan of the invention has an average molecular weight of about 80,000, about 130,000 or about 138,000. The fucoidan of the invention in particular preferably has an average molecular weight of 72,000 to 88,0000, 124,200 to 151,800 or 297,000 to 363,000.

A raw fucoidan used to produce the medium-molecular-weight fucoidan of the invention may be a raw fucoidan from any origin. Examples of the origin for the raw fucoidan include algae, for example, marine algae such as Kjellmaniella crassifolia, Japanese kelp (Laminaria japonica), Kjellmans tangles (Kjellmaniella ayrata), wakame (Undaria pinnatifida), mekabu (sporophyl of wakame), Eckronia kurome, arame (Eisenia bicyclis), kajime (Kjellmaniella crassifalia), giant kelp, Lessonia nigrescens, mozuku (Nemacystus decipiens, types of edible seaweed), okinawamozuku (Cladosiphon okamuranus), Ascophyllum nodosum, Ecklonia maxima, Durvillaea, and other brown algae in the order of Laminariales, and brown algae in the order of Chordariales, the order of Fucules, and other orders; and echinoderms such as sea cucumbers, sea urchins, and starfishes. However, the origin is not limited thereto. A preferred example of the raw fucoidan used in the invention is a fucoidan originating from okinawamozuku.

In the invention, the raw fucoidan may be a purified product, a roughly purified product, or a partially purified product, and may contain foreign substances or contaminants such as proteins, lipids, and other saccharides. For example, mozuku or any other sea algae may be used, as it is, as the raw fucoidan, or a crude extract therefrom may be used as the raw fucoidan. In the invention, the raw fucoidan may be in the form of an aqueous solution thereof. The aqueous solution of raw fucoidan may contain another material, such as a salt.

The medium-molecular-weight fucoidan of the invention may be produced by any method known in the art. In general, an raw fucoidan may be produced by subjecting an original material or raw material, such as an alga, to extracting treatment with an acidic aqueous solution. In order to produce a fucoidan having a specified average molecular weight, the pH of the aqueous solution, and conditions for the extraction, such as the extracting period and temperature may be appropriately decided. For example, as the pH of the aqueous solution is lower, the extracting temperature is higher, and the extracting period is longer, a further promotion of a decrease in the fucoidan molecular weight is attained so that the molecular weight of the resultant fucoidan becomes smaller. Alternatively, as the pH of the aqueous solution is closer to neutrality (=7.0), the extracting temperature is lower, and the extracting period is shorter, a decrease in the fucoidan molecular weight is less attained so that the molecular weight of the resultant fucoidan becomes larger. Those skilled in the art can easily decide conditions for yielding a fucoidan having a desired molecular weight through routine experiments.

Preferably, the medium-molecular-weight fucoidan of the invention can also be produced by subjecting a raw material to extraction at a low temperature and a pH close to the neutrality to yield a crude raw fucoidan having a relatively high molecular weight, and then treating the raw fucoidan to be converted into a medium-molecular-weight molecule form. The treatment into the medium-molecular-weight molecule form may be conducted in any manner usable for making the molecular weight of a saccharide low. The manner includes, but not limited to, for example, acid treatment, hydrolysis, thermal decomposition (at, for example, 80 to 100° C.), or enzyme decomposition, in particular, hydrothermal treatment.

More preferably, for the production of the medium-molecular-weight fucoidan of the invention, a hydrothermal treatment of a raw fucoidan is used. The hydrothermal treatment is generally conducted by use of subcritical water (at a temperature of 100 to 374° C. and a pressure of 0.1 to 22 MPa). The treatment can be preferably conducted according to a method described in JP-A-2008-266299, which is incorporated into the present specification by reference. The hydrothermal treatment can be generally conducted by holding an aqueous solution of the raw fucoidan in a sealed reactor at about 100 to about 180° C. for about 5 to about 20 minutes, at about 100 to about 160° C. for about 5 to about 20 minutes, or at about 100 to about 140° C. for about 5 to about 60 minutes in the state that the pH is not adjusted or is adjusted into the range of about 6 to about 12. In accordance with a target molecular weight of the medium-molecular-weight fucoidan, these conditions can easily be adjusted or selected by those skilled in the art. Although the technical scope of the invention is not restricted by any theory, the molecular weight of the medium-molecular-weight fucoidan can be adjusted by the hydrothermal treatment while the sulfate groups are kept. The following fucoidan has a very good antitumor effect: a medium-molecular-weight fucoidan in which its sulfate groups are kept in a proportion of 90% or more, preferably 95% or more, more preferably 98% or more compared with sulfate groups of the raw fucoidan.

In order to purify the medium-molecular-weight fucoidan of the invention, a purifying treatment may be conducted at any stage. In other words, a purifying treatment is conducted before and/or after the treatment of the raw fucoidan into the medium-molecular-weight molecule form, thereby making it possible to gain only a medium-molecular-weight fucoidan having specified molecular weights. At the same time, foreign substances or contaminants such as proteins, lipids, and other saccharides may be removed. The purification may be conducted in any manner known in the art. The manner is preferably gel permeation chromatography, or ultrafiltration.

The term “average molecular weight” used in the present specification denotes weight-average molecular weight (Mw), and this molecular weight may be measured by any method known in the art. Examples of this measuring method include vapor pressure osmometry, (dynamic and static) light scattering methods, size-excluding chromatography, and electrophoresis. The method is in particular preferably size-excluding chromatography. The average molecular weight of the fucoidan of the invention may be measured by, for example, size-excluding chromatography using a differential refractometer. Specific measuring conditions therefor, for example, a device, columns, a mobile phase and a temperature therefor can be appropriately selected with ease by those skilled in the art, dependently on various situations, such as the molecular weight of a subject to be measured, and the concentration of the sample.

The term “about” used in the specification means any numerical value in the range of ±10% of a numerical value to which the term is attached. For example, the term “about 10,000” means the range of 9,000 to 11,000. When numerical values are described by use of a range in the specification, it should be understood that the range mean the upper limit and the lower limit of the numerical values, all values between the limits, and the range of all the values between the limits. It can be understood that the description “1 to 10” refers also to 1, 10, 3, values of 2 to 5, and others.

The term “treat(ing) or treatment” used in the invention includes the extinction or relief of at least one symptom that is related to a state, damage or disease to be treated, or is caused thereby. In an embodiment, the treatment may be the extinction, contraction or decrease, metastasis-prevention, progression-arrest, or retardation of a proliferative disease, in particular, a cancer selected from, for example, bladder cancer, head and neck cancer, breast cancer, gastric cancer, ovarian tumor, esophageal cancer, colon cancer, rectum cancer, brain tumor, pharyngeal cancer, lymphatic cancer, reproductive urinary tract cancer, squamous cell carcinoma, cutaneous cancer, cancer of digestive organ, prostatic cancer, bone cancer, (small cell and non-small cell) lung cancer, glioma, and spleen cancer, or the extinction or relief of at least one symptom caused by the cancer.

A subject for which the medium-molecular-weight fucoidan of the invention is intended to be used is a mammal that may get or undergo a disease, disorder or state related to an excessive proliferation of cells. Examples of the subject include humans, chimpanzees, dogs, cattle, horses, pigs, sheep, goats, monkeys, cats, mice, rabbits, rats, and transgenic non-human animals. In an embodiment, the subject is a human or a mammalian domestic animal, for example, a human or a mammalian domestic animal who or that has a cancer, has a risk of having a cancer, or may latently have a cancer.

The antitumor activity of the medium-molecular-weight fucoidan of the invention can be measured by use of many assays usable in the art. Examples of the assays will be given in working examples that will be described later.

The term “pharmaceutical composition” used in the invention may be a pharmaceutical preparation suitable for being administered to a mammal, such as a human. When the compound of the invention is administered as a pharmaceutical agent to a mammal such as a human, the compound may be administered as it is, or in the form of a pharmaceutical composition containing the effective component in a proportion of, for example, 0.1 to 99.5% (more preferably 0.5 to 90%) in combination with a pharmaceutically acceptable carrier.

In general, the compound of the invention is administered in an effective amount, in particular, in an amount effective for treatment, alone or in a combination with one or more curative medicines, in any ordinarily acceptable form known in the art. The amount effective for treatment may be varied within a wide range dependently on the severity of the disease, the age and the relative healthy condition of the subject, the ability of the used compound, and other factors. In general, the dose to the whole body per day is from about 1.5 to about 15,000 mg per kilogram of the weight in terms of powdery weight, which may produce a satisfactory result. The dose per day instructed for a large mammal, such as a human, ranges from about 100 to about 10,000 mg in terms of powdery weight. According to a simple way, for example, the compound is administered, at a divided dosage, four or less times per day, or is administered in a sustained release form. A single-administration form (of the compound) suitable for oral administration contains the effective component in an amount about 25 to about 2,500 mg.

The term “pharmaceutically acceptable carrier” used in the invention may include a pharmaceutically acceptable substance, composition or vehicle that is understandable by those skilled in the art and that is suitable for the administration of the compound of the invention into a mammal. The carrier may be a liquid or solid filler, diluting agent, excipient, solvent or capsule material through which the subject pharmaceutical agent is delivered or transported from an internal organ or a region of the body to another internal organ or another region of the body. The carrier is compatible with other components of the pharmaceutical agent, and needs to be “acceptable” in the point that the carrier does not damage any patient. Examples of a substance usable as the pharmaceutical acceptable carrier include sugars, such as lactose, glucose, and saccharose; starches, such as corn starch, and potato starch; cellulose, and derivatives thereof, such as sodium carboxymethylcellulose, ethylcellulose, and cellulose acetate; powdery tragacanth; malt; gelatin; talc; excipients, such as cocoa butter, and suppository wax; oils, such as peanut oil, cotton oil, sunflower oil, sesame oil, olive oil, corn oil, and soybean oil; glycols, such as propylene glycol; polyols, such as glycerol, sorbitol, mannitol, and polyethylene glycol; esters, such as ethyl oleate, and ethyl laurate; agar; water; and ethyl alcohol. However, the substance is not limited thereto.

The pharmaceutical composition of the invention may contain additives besides the medium-molecular-weight fucoidan of the invention and the pharmaceutical acceptable carrier, the examples of the additives including a moisturizing agent, an emulsifier, a lubricating agent, a colorant, a releasing agent, a coating agent, a sweetener, a flavoring agent, a preservative, and an antioxidant. These additives themselves, and the use thereof are well known by those skilled in the art.

The composition of the invention may be a composition suitable for oral, nasal, local, buccal, sublingual, rectal, transvaginal, and/or parenteral administration(s). The composition may simply be in an unit dosage form, and may be prepared by any method well-known in the pharmaceutical field. The amount of the active ingredient that can be combined with the carrier material to provide an unit dosage form is generally a compound amount producing a curative effect. For this amount, the proportion of the effective amount ranges generally from about 1 to about 90% by weight of the total amount of the composition, preferably from about 5 to about 70% by weight thereof, most preferably from about 10 to about 30% by weight thereof.

The process for producing the composition comprises the step of mixing a medium-molecular-weight fucoidan of the invention with a carrier and one or more optional desired additives. In general, the composition of the invention is prepared by mixing the medium-molecular-weight fucoidan evenly and closely with a liquid carrier or a micronized solid carrier, or the two, and, if desired, forming the prepared mixture into a shape.

The composition of the invention that is suitable for oral administration may be prepared in a form of the following matter which contains the medium-molecular-weight fucoidan of the invention in a predetermined amount: a capsule agent, cassette, pill, tablet, troche (in which one or more flavor base agents, which are usually saccharose, and acacia or gum tragacanth, are used), powder, granule, aqueous- or non-aqueous-liquid solution or suspension, oil-in-water or water-in-oil emulsion, elixir agent or syrup, or troche (in which one or more inactive base agents, such as gelatin and glycerol, and acacia or gum tragacanth, are used).

The composition of the invention that is suitable for parenteral administration may be prepared in a formulation for an injection or infusion solution containing the medium-molecular-weight fucoidan of the invention as an effective component in a predetermined amount, for example, for an intravenous, intramuscular or subcutaneous injection or infusion solution, or for bolus administration and/or sustained release injection. The pharmaceutical composition of the invention for parenteral administration, may be a suspension, a solution or an emulsion in an oily or aqueous vehicle comprising a different formulating agent if desired, for example a suspending agent, a stabilizer and/or a dispersing agent. Alternatively, the composition of the invention may be a sterilized and freeze-dried powdery formulation, which is to be dissolved or suspended in an injectable water, such as sterilized water, immediately before the composition is used.

In another aspect of the invention, the pharmaceutical composition of the invention may contain an anti-inflammatory agent, an anti-cell-proliferation agent, a chemotherapeutic agent, an immunosuppressive agent, an antitumor agent, or a cytotoxic agent as an active ingredient besides the fucoidan of the invention.

In an additional embodiment, the invention provides a food or drink for treating and/or preventing a tumor comprising the medium-molecular-weight fucoidan of the invention. The form of the food or drink of the invention may be any form, and is not particularly limited. The amount of the medium-molecular-weight fucoidan of the invention comprised in the food or drink of the invention may be appropriately selected, considering the antitumor effect (of the fucoidan), the flavor or taste of the food or drink, and/or some other factors.

Hereinafter, the invention will be specifically described in more detail by way of working examples; however, the invention is not limited by the examples.

### Example 1

Production of Medium-Molecular-Weight Fucoidan

Okinawamozuku (purchased from fishery cooperative association of Iheya-mura in Okinawa Prefecture) was subjected to extraction with hot water at 60 to 90° C. under an acidic condition to yield a crude fucoidan. The weight-average molecular weight of the crude fucoidan was measured by use of a size-excluding chromatography using three connected “Asahipak” columns (GS520, GS320 and GS220) manufactured by Shodex Co., and a differential refractometer. As the result, the molecular weight was about 330,000.

A 5% solution of the crude fucoidan in water was subjected to hydrothermal treatment under each of conditions described in Table 1 shown below to prepare an aqueous solution of a fucoidan having the corresponding weight-average molecular weight. The resultant medium-molecular-weight fucoidans were each powderized by spray drying.

TABLE 1

| | Average molecular |
|---|---|
| Hydrothermal condition | weight of fucoidan |
| 140° C. for 60 minutes | 6,500 |
| 140° C. for 15 minutes | 32,000 |
| 140° C. for 15 minutes | 40,000 |
| 140° C. for 8 minutes | 80,000 |
| 140° C. for 7 minutes | 130,000 |
| 140° C. for 7 minutes | 138,000 |
| 120° C. for 10 minutes | 244,000 |

### Example 2

### Antitumor Effect of Medium-Molecular-Weight Fucoidan—Tumor Growth Rate

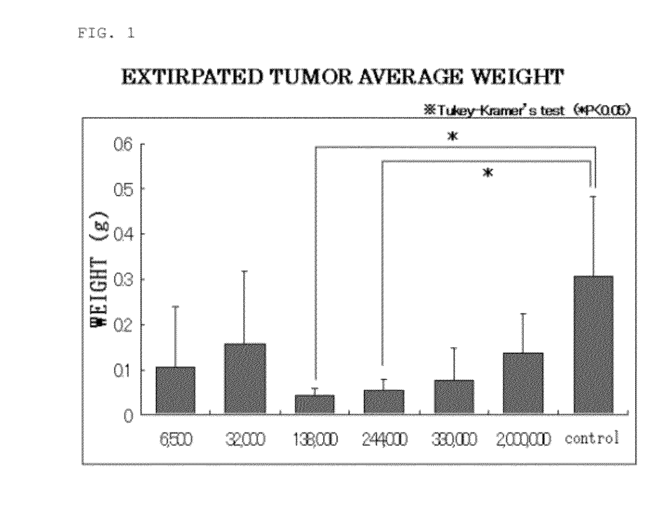

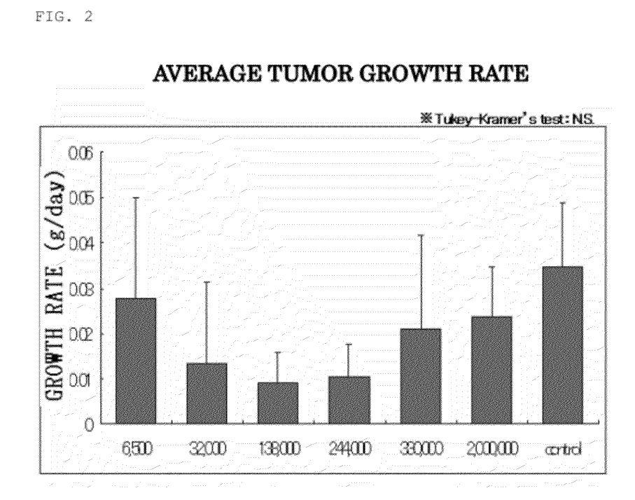

28 BALB/c mice (purchased from CLEA Japan, Inc.; weekly age: 4 to 5 weeks) were classified into the following 7 groups: a group in which a fucoidan having an average molecular weight of 2,000,000 was administered; one in which a fucoidan having an average molecular weight of 330,000 was administered; one in which a fucoidan having an average molecular weight of 244,000 was administered; one in which a fucoidan having an average molecular weight of 138,000 was administered; one in which a fucoidan having an average molecular weight of 32,000 was administered; one in which a fucoidan having an average molecular weight of 6,500 was administered; and a control group in which no fucoidan was administered. The groups were each kept for 28 days while a powdery keeping (purchased from CLEA Japan, Inc.) into which the fucoidan having the corresponding molecular weight was incorporated to give a proportion of 5% by weight was given to the group. The control group was kept while a powdery keeping into which no fucoidan was incorporated was given thereto. After the 28 days, each piece of Colon 26 (settled from The Cancer Institute Hospital of JFCR), 1 mm square, was weighed, and then the weight of a tumor at 0^{th} day was recorded. Thereafter, the pieces of Colon 26, 1 mm square, were each subcutaneously transplanted as a tumor tissue to the back of each of the mice, and further the mice were kept for 14 days while a powdery keeping into which the corresponding fucoidan was incorporated to give a proportion of 5% by weight, or a powdery keeping into which no fucoidan was incorporated was given to the mice. On the 14^{th} day from the transplant, each of the mice was killed, and a tumor therein was collected therefrom. The tumor was weighed, and then the weight of the extirpated tumor at the 14^{th} day was recorded. The tumor growth rate (g/day) was calculated from the expression of (“the extirpated tumor weight at the 14^{th} day”—“the tumor weight at the 0^{th} day”)/14. The results are shown in FIGS. 1 and 2. FIG. 2 demonstrates that the groups in which the medium-molecular-weight fucoidans, in particular, the fucoidans the average molecular weights of which were 32,000, 138,000 and 244,000, were administered were significantly lower in tumor growth rate than the control.

### Example 3

Antitumor Effect of Medium-Molecular-Weight Fucoidan/Survival Period

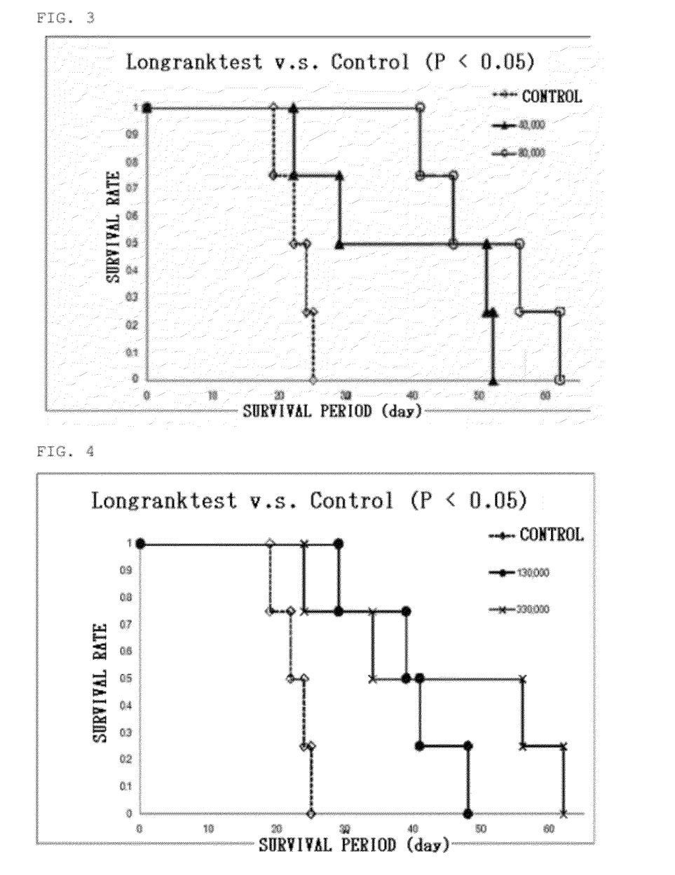

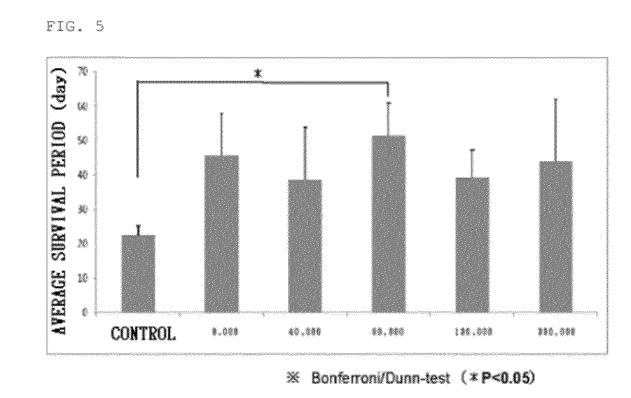

24 BALB/c mice (purchased from CLEA Japan, Inc.; weekly age: 4 to 5 weeks) were classified into the following 6 groups: a group in which a fucoidan having an average molecular weight of 330,000 was administered; one in which a fucoidan having an average molecular weight of 130,000 was administered; one in which a fucoidan having an average molecular weight of 80,000 was administered; one in which a fucoidan having an average molecular weight of 40,000 was administered; and a control group in which no fucoidan was administered. The groups were each kept for 28 days while a powdery keeping (purchased from CLEA Japan, Inc.) into which the fucoidan having the corresponding molecular weight was incorporated to give a proportion of 5% by weight was given to the group. The control group was kept while a powdery keeping into which no fucoidan was incorporated was given thereto. After the 28 days, a piece of Colon 26 (settled from The Cancer Institute Hospital of JFCR), 1 mm square, was subcutaneously transplanted as a tumor tissue to the back of each of the mice. The day when the transplant was performed was defined as the 0^{th} day. The mice were kept while a powdery keeping into which the corresponding fucoidan was incorporated to give a proportion of 5% by weight, or a powdery keeping into which no fucoidan was incorporated was given to the mice. The period until each of the mice was died was defined as the survival period thereof. The results are shown in FIGS. 3 and 4. FIGS. 3 to 5 demonstrate that the groups in which the medium-molecular-weight fucoidans were each administered, was made significantly better in survival period after the transplant of the tumor tissue than the control.

1. A medium-molecular-weight fucoidan having an average molecular weight of 40,000 to 330,000.

2. The medium-molecular-weight fucoidan according to claim 1, wherein the average molecular weight is from 40,000 to 244,000.

3. The medium-molecular-weight fucoidan according to claim 1, wherein the average molecular weight is from 80,000 to 200,000.

4. The medium-molecular-weight fucoidan according to claim 1, wherein the average molecular weight is from 130,000 to 138,000.

5. The medium-molecular-weight fucoidan according to claim 1, wherein the average molecular weight is from 72,000 to 88,000.

6. The medium-molecular-weight fucoidan according to claim 1, wherein the average molecular weight is from 124,200 to 151,800.

7. The medium-molecular-weight fucoidan according to claim 1, wherein the average molecular weight is from 297,000 to 363,000.

8. The medium-molecular-weight fucoidan according to claim 1, which is produced by hydrothermal treatment.

9. The medium-molecular-weight fucoidan according to claim 1, which is useful for treating and/or preventing a proliferative disease.

10. A pharmaceutical composition, comprising a medium-molecular-weight fucoidan according to claim 1, and a pharmaceutically acceptable carrier.

11. The pharmaceutical composition according to claim 10, which is used to treat and/or prevent a proliferative disease.

12. The pharmaceutical composition according to claim 11, wherein the proliferative disease is a cancer.

13. Use of a fucoidan according to claim 1 in preparation of a medicament for treating and/or preventing a proliferative disease.

14. A method for treating and/or preventing a proliferative disease, comprising administering a medium-molecular-weight fucoidan according to claim 1 in an effective amount to a subject for which the treatment and/or the prevention is required.

15. A food or drink useful for treating and/or preventing a tumor, comprising a medium-molecular-weight fucoidan according to claim 1.
